(19) United States
(12) Patent Application Publication
(10) Pub. No.: US 2022/0229700 A1
(43) Pub. Date: Jul. 21, 2022

(54) METHOD FOR SECURELY EXECUTING A WORKFLOW IN A COMPUTER SYSTEM

(71) Applicant: CAMELOT ITLAB GMBH, Mannheim (DE)

(72) Inventors: Andreas GOEBEL, Forst (DE); Steffen JOSWIG, Laudenbach (DE); Josef PACKOWSKI, Heidelberg (DE)

(21) Appl. No.: 17/716,755

(22) Filed: Apr. 8, 2022

Related U.S. Application Data
(63) Continuation of application No. PCT/EP2020/078331, filed on Oct. 8, 2020.

(30) Foreign Application Priority Data
Oct. 8, 2019 (EP) 19202043.6

(51) Int. Cl. G06F 9/50; H04L 9/40

(52) U.S. Cl. CPC G06F 9/5027; H04L 63/04; H04L 9/40

(57) ABSTRACT

A method is provided for securely executing a workflow in a computer system which comprises at least a distributed repository network and a number of client systems that are operatively coupled or operatively couplable to this repository network. A model of the workflow is stored in the repository network. The workflow model comprises a number of workflow steps, and execution conditions, events, tasks and notifications are stored in the workflow model for each workflow step. To execute the workflow, a workflow instance is generated from the workflow model and is stored in the repository network. The client systems execute the workflow instance stored in the repository network.

### CROSS REFERENCE TO RELATED APPLICATIONS

This application is a continuation of International Application No. PCT/EP2020/078331, filed Oct. 8, 2020, which claims priority to European Patent Application No. 19202043.6 filed Oct. 8, 2019, the contents of each of which are incorporated by reference herein.

### FIELD OF THE INVENTION

The invention relates to a computer-implemented method for securely executing a workflow in a computer system.

### BACKGROUND

It is known to execute workflows in a distributed network involving a plurality of participants who are involved in the execution of a workflow. Each of these participants can perform different steps of the workflow, wherein the workflow step can be executed on a different client system in the distributed network.

However, the disadvantage of such workflow systems is that, on the one hand, it cannot be guaranteed that the definition of a workflow, i.e. the workflow model, will not be manipulated. On the other hand, it also cannot be guaranteed that the executed workflow, i.e. the workflow instance of a workflow model, will not be manipulated. Both types of manipulation can result in a workflow not being executed as expected by participants. For example, a manipulated workflow instance can lead to

trustworthy data being disclosed, or

workflow steps being executed which should not be executed, or

data in the computer systems being manipulated.

From the document “Orlenys Lopez-Pintado et al.: ‘Caterpillar: A Business Process Execution Engine on the Ethereum Blockchain,’ Software: Practice and Experience, 2018; 00:1-45,” a system is known in which a blockchain platform is used for the execution of workflows, possibly distributed execution. Each workflow to be executed is first compiled for this purpose. The result of the compilation, i.e. an executable program code, is “installed” or stored in the blockchain as what is known as a smart contract. However, the disadvantage here is that every time the workflow is changed, it has to be recompiled and stored in the blockchain again as a smart contract. Ongoing maintenance and additions to workflows are always associated with the creation of a new smart contract.

An object of the present invention is therefore to provide a solution which, on the one hand, enables a workflow instance to be executed securely in a computer system, with both the workflow instance and the workflow model on which the workflow instance is based being protected from manipulations, and which, on the other hand, enables the workflow model to be adapted and changed flexibly without having to compile the workflow model after each change.

### SUMMARY

Accordingly, a computer-implemented method for securely executing a workflow in a computer system is provided, comprising at least one distributed repository network and a number of client systems that are operatively coupled or can be operatively coupled to this repository network, wherein
a model of the workflow is stored in the repository network,
the workflow model comprises a number of workflow steps, a predetermined workflow step being defined as an initial step which is executed as the first step when executing the workflow in the computer system, and wherein stored in the workflow model for each workflow step are
   execution conditions that must be met to execute the workflow step in the computer system,
   events (in other words information about events) recorded in the computer system during the execution of the workflow step,
   tasks (in other words information about tasks) assigned to a user, user role or client system when a workflow step is executed in the computer system, and
   notifications (in other words information about notifications) made available to a user, user role or client system when a workflow step has been successfully executed in the computer system or when a workflow step has to be executed in the computer system,
to execute the workflow in the computer system a workflow instance is generated from the workflow model, the instance representing the workflow to be executed, the workflow instance being stored in the repository network,
when executing the workflow instance in the computer system,
   first of all the initial step is executed, and then the further workflow steps are executed,
   when a workflow step is started, the events, tasks and notifications are generated (in other words according to the respective information stored in the workflow model) and stored in the repository network,
   state data are stored in the repository network for each executed workflow step, and
   a workflow application executed on at least one client system
      receives notifications from the repository network that had been stored for a predetermined user in the repository network, and
      executes a program code assigned to the workflow step for a workflow step to be executed on the at least one client system.

This avoids having to create a new smart contract for each workflow model and for each change in a workflow model, and having to store the new smart contract in the repository network. Thus, when a workflow model is changed, it is only data, i.e. data about the workflow model, that has to be stored or changed in the repository network, but no new smart contracts are required. In the case of a repository network designed as a blockchain, there is no need for a complex and expensive distribution (deployment) of smart contracts after there is a change in the workflow model.

It is advantageous if the distributed repository network is a blockchain network.

This ensures that there are no changes in the workflow model when it is stored. The workflow instance is also stored unchangeably. Manipulations of the workflow model and the workflow instances can thus be effectively prevented. By storing the state data in the repository network, unchanging logging of the execution of a workflow instance can also be achieved.

Information on a number of actions can be stored in the workflow model for a workflow step, the actions being executed when the workflow step is executed, and the actions being generated and stored in the repository network when the workflow step is started based on the information on the number of actions.

In one embodiment of the invention, it may be advantageous if
an interpreter is stored in the repository network, preferably in a repository network configured as a blockchain,
the interpreter is executed in the repository network, and
when executing the workflow instance, the interpreter
   monitors the execution of the workflow instance according to the associated workflow model,
   when a workflow step is started, generates the events, tasks and notifications and stores them in the repository network according to the associated workflow model, and
   stores the state data in the repository network for each executed workflow step.

The interpreter can be stored as a smart contract in the repository network. The advantage here is that the interpreter does not have to be changed when the workflow model changes.

It is advantageous if the program code assigned to a workflow step is stored on the client system as part of the workflow application.

However, it is particularly advantageous if the program code assigned to a workflow step is stored in the repository network. This ensures that the program code is also stored unalterably.

The state data can include a timestamp indicating the time the workflow step was executed, a user ID of the user who executed the workflow step, and a status of the workflow step.

When the program code assigned to the workflow step is executed by the workflow application, data that is necessary for executing the program code can be transmitted to the workflow application.

The transmitted data can be stored in a storage device of the client system on which the program code is executed and/or in the distributed repository network.

Data can be generated when the workflow application executes the program code assigned to the workflow step. The generated data can be stored in a storage device of the client system on which the program code is executed and/or in the distributed repository network.

It is advantageous if the data generated is stored in encrypted form in the repository network.

In one embodiment of the invention, the workflow model can comprise a first and at least a second sub-workflow, each sub-workflow comprising a number of workflow steps, and wherein a model of the first sub-workflow is generated in a first client system of the number of client systems and a model of the at least a second sub-workflow is generated in a second client system of the number of client systems, the models of the sub-workflows being combined and stored in the distributed repository network as a workflow model.

A user and/or a user role can be assigned to each sub-workflow model, with this assignment granting the user and/or the user role management rights, in particular rights to change the respective sub-workflow model.

It is advantageous if, before a workflow step is executed, the workflow application checks whether the execution conditions for executing the workflow step are met.

The execution conditions include at least one from the group consisting of
the workflow step to be executed must be a valid workflow step, wherein a workflow step to be executed is valid if preceding workflow steps on which the workflow step to be executed depends have been executed successfully,
the user who starts the workflow step to be executed must have permission to execute the workflow step, the permissions being stored in the distributed repository network, and
combinations of these.

### BRIEF DESCRIPTION OF THE DRAWING

Further details and features of the invention will become apparent from the following description when taken in conjunction with the figures. In the figures:

FIG. 1 is a block diagram of a computer system for securely executing a workflow;

FIG. 2 is a block diagram which describes the method for securely executing a workflow in more detail;

FIGS. 3 and 4 are an example of a workflow model;

FIG. 5 is an example process for creating a trustlet;

FIG. 6 is a block diagram which describes the method for the secure validation of trustworthy data is described in more detail;

FIG. 7 is a flowchart for describing a concrete example of the method for securely validating trustworthy data; and

FIG. 8 is a block diagram which describes a further embodiment of the method for securely executing a workflow.

### DETAILED DESCRIPTION OF THE PREFERRED EMBODIMENTS

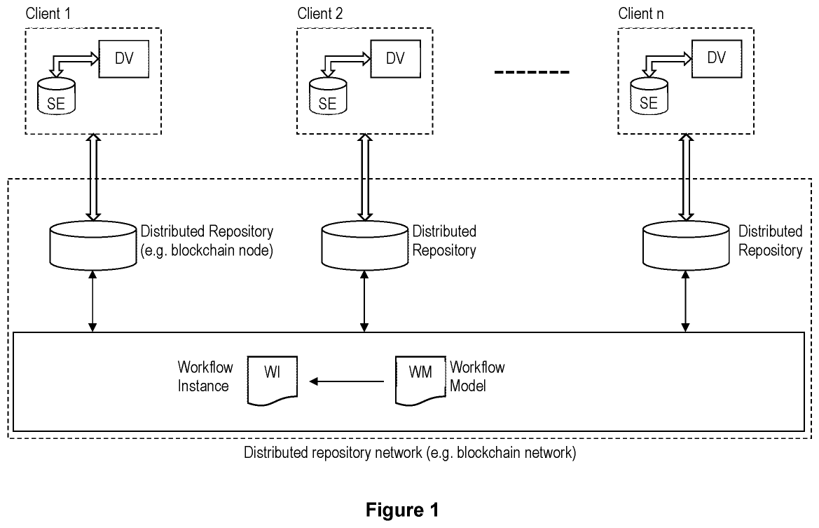

FIG. 1 shows a block diagram of a computer system for securely executing a workflow. The computer system includes a distributed repository network which is preferably configured as a blockchain network consisting of a plurality of distributed blockchain nodes. Furthermore, the computer system comprises a number of client systems (Client 1, Client 2, . . . , Client n) which are operatively coupled to or are operatively couplable to the distributed repository network. The client systems preferably each include a data processing system DV, each of which in turn includes at least one storage device SE (local storage device).

The blockchain nodes can each be assigned to the individual client systems, i.e. the data from these blockchain nodes can be stored in a respective memory device of the client systems. Alternatively, the blockchain nodes can also be kept remote from the respective client systems, with the client systems being able to access the blockchain nodes via a communication network (e.g. via the Internet).

A model of a workflow (also referred to below as a workflow model) is generated on one of the client systems and stored in the distributed repository network. The workflow model is then available for execution by the client systems.

To execute the workflow defined by the workflow model, a workflow instance is generated in the computer system from the workflow model. This workflow instance is then in turn stored in the distributed repository network. Execution conditions and the exact process when executing a workflow or a workflow instance are described in more detail below.

When executing a workflow, i.e. a workflow instance, data can be generated which in turn can be stored in the distributed repository network. Alternatively or additionally, the data generated when executing a workflow instance can also be stored in the local storage devices SE of the client systems.

Furthermore, for executing a workflow instance, data can also be made available which can also be stored in the distributed repository network and/or in the local storage devices SE of the client systems.

Workflow applications, as they are called, are executed on the client systems that are involved in the execution of a workflow instance. A predetermined program code is used by the workflow application for executing each workflow step to be executed. The program code can be a so-called chain code or a smart contract.

The program code for a workflow step can be stored in the respective client system, for example as part of the respective workflow application. Alternatively, the program code can also be stored in the distributed repository network and transferred to the respective client system or to the respective workflow application for execution. In one embodiment of the invention, the program code can also be brought for execution on a blockchain node.

A workflow step can include multiple actions. In this case, the program code of the workflow step can include a plurality of program code sections, with each action of the workflow step being assigned a program code section.

A workflow instance can contain a plurality of workflow steps, each workflow step able to be executed on a different client system or by a different workflow application.

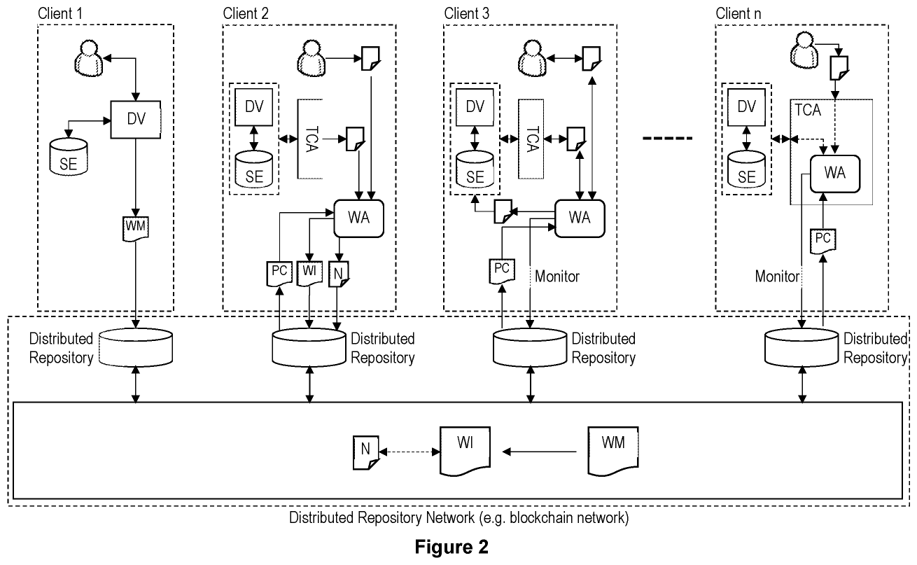

FIG. 2 shows a block diagram which describes the method for securely executing a workflow in more detail.

In the example shown in FIG. 2, four people are involved in the modeling or in the execution of a workflow, with each person being assigned to a client system.

In principle, the entire process can be divided into 3 main steps:
Main step 1: Creating the workflow model;
Main step 2: Creating a workflow instance, i.e. starting a workflow based on a workflow model; and
Main step 3: Executing the workflow instance.

### Main Step 1: Creating the Workflow Model

In the example shown in FIG. 2, the main step 1, i.e. the creation of a workflow model, is carried out on the first client system (Client 1). For this purpose, software for modeling workflows can be executed on the data processing device DV of the first client system, the desired workflow model able to be created using the software.

A process that is described with a workflow model usually consists of a plurality of process steps, a plurality of participants able to be involved in the process.

A workflow model describes a number of workflow steps, wherein a process step in the workflow model can be represented by a plurality of workflow steps. In the simplest case, a process step is represented by a workflow step. In addition, a workflow model describes interactions with users (participants involved in the process) and what data is required for the execution of the workflow and what data is generated when the workflow is executed.

The generated workflow model is stored in the distributed repository network, preferably a blockchain network.

According to embodiments of the invention, at least the following is stored or defined in the workflow model for each workflow step:

Execution conditions that must be met to execute the respective workflow step in the computer system. For example, these can be authorizations that a user must have in order to execute the workflow step. Another execution condition could define, for example, which previous workflow steps must be completed successfully in order to be able to execute the workflow step. Yet another execution condition could for example define which data must be present in the distributed repository network and/or in the local storage device of the client system on which the workflow step is to be executed in order to be able to execute the workflow step.

The execution conditions for executing the workflow or the workflow instance itself are defined by the execution conditions of the initial step (workflow step that is executed as the first step when the workflow instance is started) of the workflow instance.

Events recorded in the computer system when the workflow step is executed. Events can be, for example, a change in the status of the executed workflow instance or the generation of data by the workflow instance. It is advantageous if the events generated when the workflow instance is executed are stored in the distributed repository network. The events stored in the distributed repository network can then be used as an unchanging audit trail for the respective workflow instance.

Tasks assigned to a user, user role, or client system when a workflow step is executed on the computer system. As soon as a task has been executed when a workflow instance is executed, the next workflow step or, if available, the next action within the workflow step is triggered.
a) A task can include a workflow step to be performed.
b) Alternatively, a task may include an action that must be performed by a user external to the computer system and that must be confirmed to be performed in the computer system. The acknowledgment can be digitally signed and stored in the distributed repository network.
c) In yet another alternative, a task may include an action to be performed (without user interaction) by a client system, for example. For example, a task may consist of the client system performing a function in an external computer system. After successful execution, the client system can confirm the execution, the confirmation preferably being digitally signed and stored in the distributed repository network.

Notifications provided to a user, user role, or client system when a workflow step has been successfully executed on the computer system or when a workflow step needs to be executed on the computer system. When a workflow instance is executed, the notifications are stored in the distributed repository network.

It is advantageous if the notifications are stored in encrypted form in the distributed repository network, advantageously with the public key of the instance (user, user role or client system) for which the notification is intended. The public keys required for this can also be stored in the distributed repository network.

The workflow applications executed on the client systems can monitor the distributed repository network for new notifications. For example, after a workflow step of a workflow instance has been successfully executed on a first client system, a notification for a second client system can be generated and stored in the distributed repository network. With this notification, the second client system can, for example, be informed that the next workflow step can now be executed. Once the second client system “recognizes” this notification, it can proceed to the next workflow step.

Instead of monitoring the distributed repository network for newer notifications, the generated notifications can also be sent directly to the appropriate recipients by the distributed repository network, for example as an e-mail. The notifications can also be stored in the distributed repository network in this case.

Furthermore, information on a number of actions can be defined and stored in the workflow model for each workflow step, and the actions can be carried out when the workflow step of a workflow instance is executed.

In a further embodiment of the invention, provision can be made for storing so-called rollback information or rollback actions in the workflow model with which actions and/or workflow steps already executed when a workflow instance is executed can be reversed.

In one embodiment of the invention, it is provided that the software for modeling workflows and/or the distributed repository network validate a generated workflow model so that a workflow instance can only be generated if the workflow model has been successfully validated. For example, it can be validated whether all execution conditions specified in the workflow model are valid.

A workflow model can be stored in multiple versions in the distributed repository network. It is advantageous here if a workflow instance can only be created for the most current version of the workflow model. If a workflow instance is in execution that was started before a new version of the workflow model was stored in the distributed repository network, then this workflow instance will continue to be executed according to the older version's workflow model.

In the workflow model, a predetermined workflow step can be defined as an initial step. The initial step of a workflow is the step that is executed as the first workflow step when a workflow instance is started or executed. Alternatively, the first workflow step in the workflow model is the initial step.

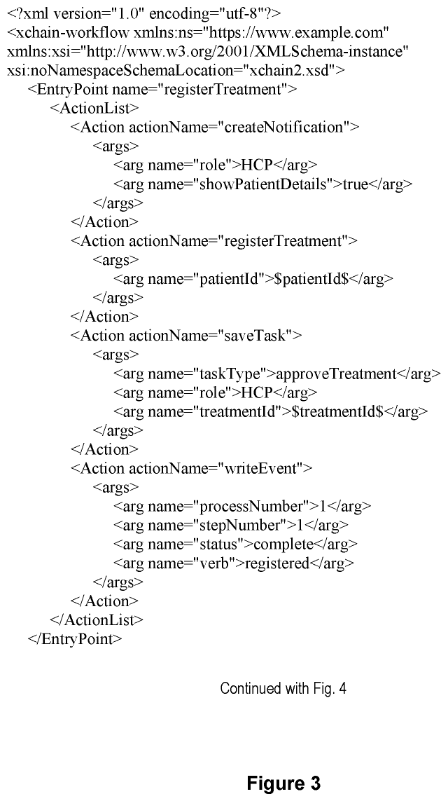

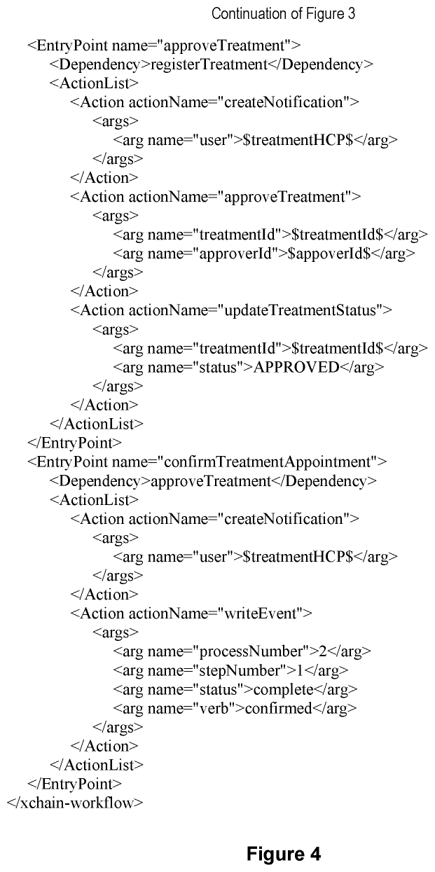

In one embodiment of the invention, the workflow model can be generated as an XML file and stored in the distributed repository network. An example of a workflow model in XML, notation is shown in FIGS. 3 and 4.

The nodes <EntryPoint> represent the workflow steps.

The <Dependency> node below the <EntryPoint> node represents execution conditions that must be met before the respective workflow step can be executed so that the workflow step can be executed. For example, the <Dependency>registerTreatment</Dependency> node of the workflow step “approveTreatment” (<EntryPoint name=“approveTreatment”>) indicates that the workflow step “registerTreatment” (<EntryPoint name=“registerTreatment”>) must have been successfully completed before the workflow step “approveTreatment” can be executed.

The <Action> nodes below the <ActionList> node represent the actions that are being executed or are to be executed when executing the workflow step of a workflow instance. With the nodes <arg>, a number of arguments can be defined for the respective action. Values of arguments that are set between two $'s (e.g. $patientId$) represent dynamic values that are set during execution of the workflow instance (e.g. by the workflow application). Such dynamic values can either be stored in the distributed repository network and read out from the distributed repository network during the execution of the respective workflow step and made available to the client system or the workflow application that executes the workflow step. Alternatively, such dynamic values can also be stored in the local memory device of the client system executing the workflow step, with the respective workflow application being able to read out the corresponding value from the local memory device in order to execute the workflow step.

Exemplary actions are <Action actionName=“createNotification”>, <Action actionName=“saveTask”> and <Action actionName=“writeEvent”>.

The action “createNotification” generates a notification and stores it in the distributed repository network, with the “user” argument specifying the user or the “role” argument specifying the user role for which the notification is intended.

The action “saveTask” generates tasks which are assigned to a user or a user role, where the “user” argument is the user or the “role” argument is the user role for which the task is intended.

The action “writeEvent” stores events occurring in the computer system when executing the workflow step in the distributed repository network. The action

```
<Action actionName=“writeEvent”>
  <args>
    <arg name=“processNumber”>2</arg>
    <arg name=“stepNumber”>1</arg>
    <arg name=“status”>complete</arg>
    <arg name=“verb”>confirmed</arg>
  </args>
</Action>
```

indicates, for example, that the action specified by “<arg name=“stepNumber”>1</arg>” of the workflow step specified by “<arg name=“processNumber”>2</arg>” is completed and confirmed. In the case of a distributed repository network designed as a blockchain, the current status of a workflow instance can be stored while all previously stored statuses remain unchangeable.

Ultimately, with the action “writeEvent” the status of the executed workflow instance is stored in the distributed repository network. If a plurality of statuses are stored for a workflow instance in the distributed repository network, then the last stored status indicates the current status of the workflow instance.

The workflow model shown in FIGS. 3 and 4 describes a simple workflow for treating a patient. It consists of three workflow steps: “registerTreatment,” “approveTreatment” and “confirmTreatmentAppointment.”

The workflow step “registerTreatment” is the initial step that is executed when the workflow instance is created.

The “registerTreatment” workflow step is used to initialize the treatment and includes four actions:

“createNotification”—this is used to create a notification for the user role “HCP” when the workflow step is executed, with the notification containing details about the patient. The action “createNotification” is the first action that is executed when executing the initial step. This means that when a workflow instance is generated, a notification for the “HCP” user role is first created and stored in the distributed repository network.

“registerTreatment”—this generates a treatment data record and stores it in the local storage device or in the distributed repository network. The treatment data record is preferably stored in encrypted form. Alternatively, an encrypted hash value of the treatment data record may be stored in the distributed repository network, while the treatment data record itself is stored in the local storage device of the client system where the treatment data record is generated. Furthermore, the treatment data record is assigned to a patient data record, with only a unique identifier of the patient data record being stored in the distributed repository network and being assigned to the treatment data record.

“saveTask”—this generates a task and assigns it to a user or a user role for completion. In the example shown in FIG. 3, the task “approveTreatment” is generated and assigned to the user role “HCP.” An identifier for the treatment or the treatment data record (treatmentId) is transmitted to this action as a further argument.

“writeEvent”—this generates an event with which the execution of the workflow step is logged. Based on the generated events, the workflow application can, for example, check whether a certain workflow step has been completed correctly before another workflow step is executed.

The workflow step “registerTreatment” is followed by the workflow steps “approveTreatment” and “confirmTreatmentAppointment.” Each of these two workflow steps also has a <Dependency> node that indicates which workflow step the respective workflow step depends on. For example, the “approveTreatment” workflow step depends on the “registerTreatment” workflow step, i.e. the “registerTreatment” workflow step must have been fully and correctly executed before the “approveTreatment” workflow step can be executed. These conditions can be checked by the respective workflow application.

The other nodes of the two workflow steps “approveTreatment” and “confirmTreatmentAppointment” correspond—adjusted accordingly—to the corresponding nodes of the workflow step “registerTreatment.”

As described above, the workflow model WM generated on the client system 1 or the corresponding XML file is stored in the distributed repository network. The workflow model is then available for execution, alternatively after validation, i.e. workflow instances of the workflow model can be generated and executed.

### Main Step 2: Creation of a Workflow Instance

The main step 2, i.e. the generation of a workflow instance based on the previously generated workflow model, is executed on the second client system (Client 2) in the example shown in FIG. 2. For this purpose, a workflow application WA is provided on the second client system, which generates a workflow instance WI of the workflow model WM stored in the distributed repository network upon request by the user of the second client system, and the generated workflow instance is stored in the distributed repository network.

The workflow instance is generated by executing the initial step defined in the workflow model. The initial step can be the first workflow step in the workflow model. The initial step is executed by the workflow application.

To execute the initial step, the execution conditions stored in the workflow model for this workflow step must be met. For example, the user or user role that wants to execute the initial step, and thus generate the workflow instance, must have the appropriate authorizations. The workflow application can check the corresponding execution conditions, in particular the authorizations.

When the workflow instance is generated, an image of the workflow model is generated and stored in the distributed repository network. The image of the workflow model can be a JSON document, for example, in which only the name and, alternatively, the version of the workflow model, the names of the workflow steps, the names of the actions and the statuses of the workflow steps are stored. This way, the workflow instances can be stored in a much more compact way than the associated workflow model. The more compact (i.e. more space-saving) storage of the workflow instance is particularly important because any number of workflow instances can be generated for a workflow model, all of which must be stored in the distributed repository network. The status of a last stored workflow instance indicates where the workflow instance is currently located as it is being executed.

When executing, or in order to execute, the initial step, the workflow application WA of the second client system (Client 2) generates a notification N and stores it in the distributed repository network. Generating notification N is the first action to be taken in the initial step. In the example shown in FIG. 3, the action “createNotification” of the workflow step “registerTreatment” is the first action to be executed.

The notification is assigned to the workflow instance, and alternatively to a workflow step, in the distributed repository network. The notification also specifies the user or the user role for which the notification was generated. In the example given, the notification is intended for the “HCP” user role.

To generate the notification N, the workflow application WA uses a program code PC which is stored in the distributed repository network and is assigned to the “createNotification” action. The workflow application reads this program code from the distributed repository network and executes it on the client system (Client 2). Alternatively, the program code PC can also be stored in the local storage device SE of the client system and can be read out from there for execution. In yet another alternative, the PC can also be part of the workflow application WA.

Then the status of the workflow instance is stored in the distributed repository network. In the present example, after the first action has been carried out, the status includes data indicating that the workflow instance is now in the first workflow step and that the second action within the first workflow step is to be carried out next.

This completes the generation of the workflow instance.

### Main Step 3: Executing the Workflow Instance.

After the workflow instance has been successfully generated (i.e. after the initial step has been executed), the other workflow steps defined in the workflow model associated with the workflow instance or the other actions provided in the initial step can be executed.

The further workflow steps or the further actions provided in the initial step can be carried out by different client systems or by workflow applications of different client systems. According to the example shown in FIG. 2, these further workflow steps or the further actions provided in the initial step are carried out by the client systems Client 3 to Client n. However, it is also possible according to embodiments of the invention that further workflow steps or further actions of the initial step are carried out by the first client system (Client 1) on which the workflow model was generated and/or by the second client system (Client 2) on which the workflow instance was created. Where which workflow steps are specifically executed or brought for execution depends primarily on which users or which client systems the respective workflow steps are assigned to.

In the example shown in FIG. 3/FIG. 4, the workflow step “approve treatment” is assigned to the user role “HCP,” as defined in the node

```
<Action actionName=“saveTask”>
  <args>
    <arg name=“taskType”>approve treatment</arg>
    <arg name=“role”>HCP</arg>
    <arg name=“treatmentId”>$treatmentId$</arg>
  </args>
</Action>
```

of the workflow model.

If only the user of the third client system (Client 3) is assigned to the “HCP” user role, then the workflow step “approve treatment” can also only be executed on the third client system or by the workflow application of the third client system (because the workflow application of the third client system executes the corresponding check of the execution conditions).

According to the example of a workflow model shown in FIG. 3/FIG. 4, the next action to be executed after the generation of the workflow instance is the action “registerTreatment,” as in the node

```
<Action actionName=“registerTreatment”>
  <args>
    <arg name=“patientId”>$patientId$</arg>
  </args>
</Action>
```

of the workflow model.

For the following description, it is assumed that the further workflow steps or the further actions provided in the initial step are executed by or on the client systems Client 3 and/or Client n or by the workflow applications of these client systems.

Furthermore, it is assumed for the following description that the first workflow step “registerTreatment” (<EntryPoint name=“registerTreatment”>) has already been executed completely and successfully.

With the “saveTask” action of the first workflow step “registerTreatment,” a task was generated and assigned to the “HCP” user role for completion. In this example, the task is to execute the workflow step “approveTreatment.”

With the action ““writeEvent” of the first workflow step “registerTreatment,” the current status of the workflow instance was stored in the distributed repository network.

After completion of the first workflow step “registerTreatment,” the first action (createNotification) of the second workflow step is executed. The “createNotification” action generates a notification N for the user specified in the “user” argument. This notifies the user that the workflow step “approveTreatment” can now be executed.

The generated notification can be transmitted from the client system on which the notification was generated directly to a specific client system or to the specified user, for example in the form of an e-mail.

Alternatively, the generated notification can be stored in the distributed repository network and the distributed repository network can transmit the notification to a specific client system or to the specified user.

In yet another alternative, the notification can be stored in the distributed repository network and the client systems or the workflow applications WA executed on the client systems can monitor the distributed repository network for notifications. For this purpose, the workflow applications can query the distributed repository network for existing notifications at regular intervals. It can be advantageous here if a workflow application only requests notifications that are intended for the respective client system or for the user of the client system.

Irrespective of the way in which the notification is transmitted, it can be advantageous to encrypt the notification and, if the notification is stored in the distributed repository network, to store the notification in encrypted form.

After the client system has received a notification or if there is a notification for the client system in the distributed repository network, the client system or the respective workflow application can execute the corresponding workflow. In the example shown here, the user “treatmentHCP” is informed using the notification that the workflow step “approveTreatment” can now be executed.

The “approveTreatment” workflow step can then be executed by the workflow application of the corresponding client system. The first action of the “approveTreatment” workflow step to be executed by the client system is the “approveTreatment” action (<Action name=“approveTreatment”>), i.e. the first action after the “CreateNotification” action.

In order to execute the “approveTreatment” action, the workflow application can bring for execution a program code that is assigned to the “approveTreatment” action. The corresponding program code PC can be stored as a smart contract or as chain code in the distributed repository network and can be transferred to the corresponding client system for execution. Alternatively, the corresponding program code can also be stored in the memory device SE of the respective client system.

Before or during execution of the program code assigned to the “approveTreatment” action, it can be provided to check whether the conditions for executing the action are met.

One of the conditions is defined in the workflow model, namely by the “<Dependency>registerTreatment</Dependency>” node. This node indicates that the workflow step “approveTreatment” cannot be executed until the workflow step “registerTreatment” has been successfully completed. The workflow application can use the status of the workflow instance to determine whether the workflow step “registerTreatment” has been successfully completed. For example, as the last action of the “registerTreatment” workflow step, the “writeEvent” action is executed, which stores the successful completion of the “registerTreatment” workflow step as a status in the distributed repository network. If this status is present in the distributed repository network, then the condition of the “<Dependency>registerTreatment</Dependency>” node can be positively validated by the workflow application and this execution condition is met.

Another condition that must be met for the execution of the “approveTreatment” workflow step can be the corresponding authorization of the executing user. According to the example shown in FIG. 3/FIG. 4, the task “approveTreatment” for the user role “HCP” was generated in the first workflow step “registerTreatment” using the action “saveTask.” This means that the “approveTreatment” workflow step can only be executed by a user who is assigned the “HCP” role. This assignment can be checked by the workflow application.

In one embodiment of the invention, it may be necessary for the workflow application to require additional data for executing the program code. Such data can be stored in the storage device SE of the client system or in the distributed repository network. Alternatively or additionally, such data can also be provided by the user. If such data is stored in the distributed repository network, it can be advantageous if this data is cryptographically encrypted or at least signed. The encryption of the data represents an implicit condition for the execution of the respective workflow step—because if the data cannot be decrypted, the workflow step cannot be executed successfully either. This means that only those instances (user, role, workflow application) that have the appropriate key for decrypting the data can successfully execute the relevant workflow step.

Furthermore, it can be provided that the workflow application that executes the program code generates data that must then be stored. This data can be stored in the local storage device SE of the respective client system or in the distributed repository network. If the data is stored in the distributed repository network, it can be advantageous if this data is stored in encrypted form, for example with a public key of that entity (user, role, workflow application) which must then be able to read the data.

Alternatively, instead of the data itself, hash values of the data can also be stored in the distributed repository network, for example when the data itself is not required. For example, it is sufficient if a first workflow step calculates a hash value for patient data and stores it in the distributed repository network so that a second workflow step can assign treatment data in the distributed repository network to the patient data. The treatment data is then hashed in the distributed repository network. The patient data itself can thus only be stored in a specific client system and thus remain protected.

A so-called Trusted Computing Appliance TCA can be provided for the workflow application to access the data stored in the client system or for storing data in the client system. A Trusted Computing Appliance TCA is a trustworthy and specially secured environment of the client system in which one or more trustworthy software components are being executed. The exchange of data between the workflow application and the storage device of the client system can be handled via the Trusted Computing Appliance TCA or via the trustworthy software components of the Trusted Computing Appliance TCA.

The source code of the trustworthy software component, also referred to below as a trustlet, is preferably verified by all those involved in the system.

In order for the workflow application to query data that is stored in the client system, the trustlet can accept a corresponding request from the workflow application. The trustlet then executes the query and passes the queried data to the workflow application.

It can be advantageous here if the trustlet signs the read-out data with a private key assigned to the trustlet and makes the signed data available to the workflow application. The workflow application can then validate the signature of the data with the trustlet's public key and only process the data further if the validation is successful.

If the signed data transmitted to the workflow application are stored in the distributed repository network, for example because they are required in a subsequent workflow step, it is advantageous if the data are stored in signed form in the distributed repository network. The trustlet's public key is also stored in the distributed repository network. This means that workflow applications executed on another client system can also validate the signature of the data using the public key of the signing trustlet.

In one embodiment of the invention, each trustlet can be assigned the same cryptographic key pair, so that only one public key needs to be stored in the distributed repository network. In a further embodiment of the invention, however, it is possible for each trustlet or at least some trustlets to be assigned different cryptographic key pairs, with the respective public keys being stored in the distributed repository network.

In a further refinement of the invention, the Trusted Computing Appliance TCA can have a storage device (a so-called private storage device of the TCA), which is therefore also present in a trustworthy and specially secured environment of the client system. It is advantageous here if only the trustlet can access this private memory device of the TCA. In this embodiment of the invention, the trustlet can read out data from the private storage device of the TCA in addition to data from the storage device SE of the client system. It is advantageous here if the data read out of the private memory device of the TCA is not transferred to the workflow application. A check can be carried out for the data read out of the private memory device of the TCA as to whether they meet predetermined conditions. The result of this check can be transferred to the workflow application without having to transfer the data to the workflow application itself. The result of this check can be digitally signed before being transferred to the workflow application.

Conversely, the trustlet can also store data that the trustlet receives from the workflow application or reads out from the private storage device of the TCA in the storage device SE of the client system or transmit it to the data processing system DV of the client system. In this case, too, the data can be digitally signed by the trustlet, and the signature of the data can be validated, for example by the data processing system DV of the client system, before being stored in the storage device SE of the client system.

In a further embodiment of the invention, the workflow application itself can be a trustlet which is executed in the Trusted Computing Appliance TCA (see Client n in FIG. 2). In this case, the program code that is required to execute the workflow steps can be part of the trustlet. Alternatively or additionally, the program code can also be stored in the distributed repository network. Like the trustlet itself, the program code stored in the distributed repository network is verified by all participants in the system and stored in the distributed repository network with a digital signature. The workflow application designed as a trustlet can then read the program code required for executing a workflow step from the distributed repository network and bring it for execution in the trusted computing appliance TCA. The workflow application can check whether the signature of the program code is valid before the program code is executed.

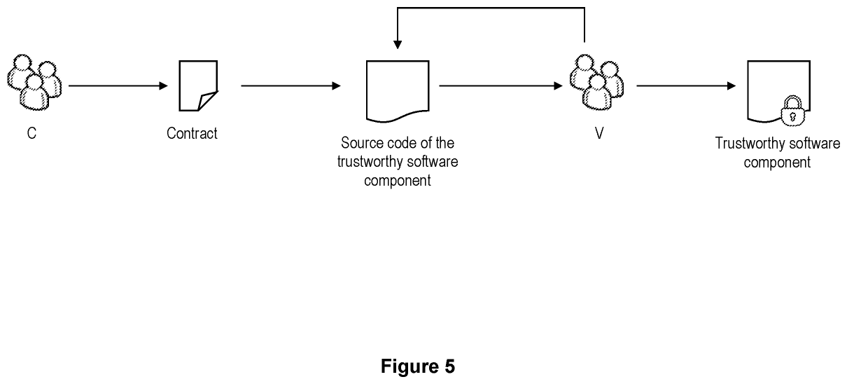

FIG. 5 shows an exemplary sequence for generating a trustworthy software component (trustlet).

A group of proprietors or owners C of trustworthy data agree on a reference document, such as a contract, in which the essential points, so-called checkpoints, for validating trustworthy data are specified.

The source code of the trustlet is created based on this reference document or on the checkpoints contained in the reference document. The source code of the trustlet is then checked or verified by one or more people V. Should errors, discrepancies or deviations from the checkpoints contained in the reference document arise, the source code of the trustlet will be adjusted and then checked or verified again. This process is carried out until the source code of the trustlet no longer shows any deviations from the checkpoints contained in the reference document and the source code of the trustlet is therefore considered verified. The trustlet is then generated from the source code of the trustlet, for example in the form of an executable program.

At this point, the trustlet can be digitally signed. In an alternative embodiment of the invention, the trustlet can then be digitally signed when it is installed on a server system in a trustworthy and secure environment of the server system. In the alternative case, the digital signature for the trustlet can be generated with a private key assigned to the server system. If each server system has different private keys, different digital signatures can be generated by server and trustlet, even if one and the same trustlet is installed on a plurality of server systems.

According to embodiments of the invention, the digital signature is stored in a distributed repository network, for example in a blockchain network. The trustlet installed on the server system can thus generate data, sign the generated data with its private key and transmit the signed data to a client system, for example. The client system can then use the server system's public key stored in the distributed repository network to check the signature of the data and, for example, only process the data further if the signature check is successful.

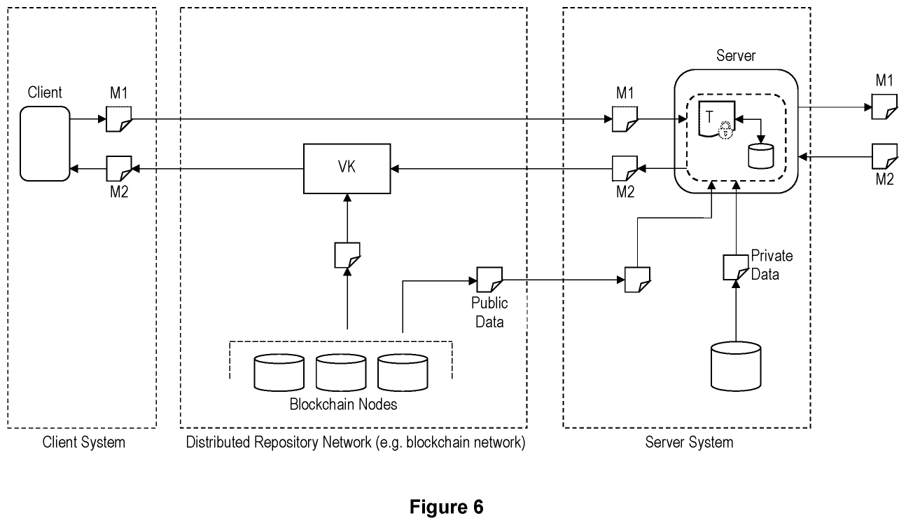

FIG. 6 shows a block diagram which is used to describe the method for securely validating trustworthy data in more detail.

FIG. 6 shows a client system, a distributed repository network operatively coupled to the client system, and a server system coupled to the distributed repository network. The distributed repository network is preferably a blockchain network that can be formed from a plurality of distributed blockchain nodes.

The server system essentially comprises a server that provides a trustworthy and secure environment (Trusted Execution Environment). The Trustlet T is installed in this trustworthy and secure environment of the server. The Trustlet T is executed exclusively in this trustworthy and secure environment.

In one embodiment of the invention, a storage device can be present in the trustworthy and secured environment, the storage device being operatively coupled to the trustlet. Preferably only the trustlet has read and write access to this memory device. However, the trustlet can also access a memory device SE of the server system that is outside the trustworthy and secure environment, with only read access being shown in FIG. 6.

When installing the trustlet in the trustworthy and secure environment of the server, the trustlet is digitally signed or a digital signature of the trustlet is generated. The trustlet's digital signature is then stored in the distributed repository network.

A first validation request message M1 is generated on the client of the client system, which is stored in the distributed repository network and is transmitted to the server of the server system. The first validation request message M1 includes information about which data is to be validated by the server of the server system or which data is to be queried by the server. For example, the first validation request message M1 can be used to find out from the server of the server system whether certain data is stored in the server system, either in the storage device provided in a secure and trustworthy environment or in the storage device SE of the server system.

The server of the server system accepts the first validation request message M1 and transmits it to the trustlet T executed in the trustworthy and secure environment.

Based on the first validation request message, the trustlet T reads out a number of data records from the storage device SE and/or from the storage device present in the trustworthy and secure environment. After the data records have been read out, the trustlet T generates a first validation response message M2 and inserts the data records read out into this response message.

In one embodiment of the invention, it can be provided that the trustlet checks whether at least one data record that has been read out meets predetermined conditions. The at least one read out data record can also be or include an aggregate of data records. In this case, the trustlet can insert information into the validation response message about which of the read-out data sets or the aggregate of data sets meet the predetermined condition, without having to insert the data sets themselves into the validation response message M2.

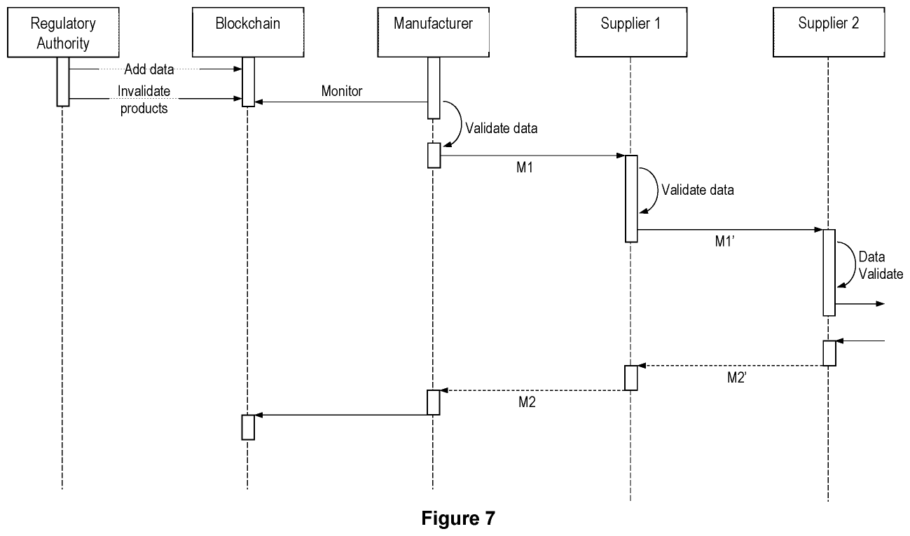

The conditions that are checked or examined here can be arbitrary. A concrete example of this will be described with reference to FIG. 7.

In one embodiment of the invention, the validation request message M1 can contain references to data stored in the server system (in the storage device SE or in the storage device present in the trustworthy and secure environment) and/or references to data stored in the distributed repository network. To process the validation request message M1, the trustlet can read out both data from the distributed repository network and data stored in the server system.

The first validation response message M2 is stored in the repository network and transmitted to the client system. It is advantageous here if both the first validation request message M1 and the first validation response message M2 are stored in encrypted form in the repository network.

In a particular embodiment of the invention, the first validation response message M2 may be digitally signed by the server or by the trustlet, with the signed first validation response message being stored in the distributed repository network. The signature of the first validation response message M2 can now be validated in the repository network using a public key of the server or of the trustlet. The server's or trustlet's public key is also stored in the distributed repository network. If the validation is positive, the validation response message can be transmitted to the client system or to the client. The validation can be performed by a validation component VK executed in the distributed repository network.

This type of validation of the validation response message M2 can also be carried out by the client itself. That is, the digitally signed first validation response message is stored in the distributed repository network and transmitted to the client. The client then validates the signature of the validation response message using the public key of the server or trustlet stored in the distributed repository network. If the signature is validated positively, the client system or the client can further process the first validation response message. Otherwise, the first validation response message can be discarded.

Accordingly, trustworthy data can be stored on the server system, i.e. in the storage device SE of the server system or in the storage device of the trustworthy and secure environment of the server, while non-trustworthy data can be stored in the distributed repository network. With the method according to embodiments of the invention it is thus possible to validate trustworthy data stored on the server system without the data itself being disclosed or else with only those data being disclosed which are not trustworthy anyway since they are stored in the distributed repository network, for example. A corresponding example of this is described in more detail below with reference to FIG. 7.

If the server of the server system cannot fully answer the validation request message M1 itself, it can be provided that the server system generates a second validation request message M1′ based on the first validation request message M1 and transmits this to another server system, also storing it in the distributed repository network. The additional server system can then correspondingly generate a second validation response message M2′ and store it in the distributed repository network and also transfer it to the server system. In this case, the server system represents a client system in relation to the other server system. The additional server system can also have a server with a secure and trustworthy environment, in which a trustlet is also executed. In this way, a hierarchical or multi-level validation of trustworthy data can be implemented.

FIG. 7 shows a flow chart for a multi-level or hierarchical validation of trustworthy data.

The process is described using an example in which a manufacturer of a product, such as a cosmetic product, has to specify certain ingredients on the packaging or on the label. These ingredients, which must be declared, are specified by a regulatory authority. The manufacturer of the cosmetic product uses ingredients known to him for the production and buys certain components of the cosmetic product from a first supplier, the manufacturer not knowing the exact composition of these components. The first supplier can in turn buy components from a second supplier, the first supplier not knowing the composition of the components obtained from the second supplier.

In order for the manufacturer of the cosmetic product to be able to specify all ingredients that are subject to declaration, the manufacturer of the cosmetic product must inquire about all ingredients that are subject to declaration from all suppliers in the entire supply chain. In the prior art, such a query may take many weeks or even months, since all suppliers have to be queried manually. In the worst case, the delivery of the manufactured cosmetic products may have to be stopped or postponed. In addition, the suppliers have no interest in disclosing the composition of the components supplied. Rather, the suppliers only want to disclose the parts of the components that are subject to declaration.

With the method according to embodiments of the invention for validating trustworthy data, it is possible, on the one hand, for only untrustworthy data (in this example, the ingredients that are subject to declaration) to be disclosed. On the other hand, the entire validation process can be reduced to a few minutes or even a few seconds, effectively preventing delivery delays.

Data on the substances or ingredients regulated by the regulatory authority are stored in the distributed repository network or in the blockchain. All entities involved in the validation process (manufacturers and suppliers) can access the data stored in the blockchain. The regulatory authority also registers products that contain at least one ingredient that must be declared.

As soon as data of a new ingredient subject to declaration are added to the distributed repository network or the data of an ingredient already added to the distributed repository network are changed by the regulatory authority, the data in the distributed repository network of the products which have such an ingredient subject to declaration which is either new or changed are marked as invalid in the distributed repository network.

The manufacturer of the cosmetic product monitors the data stored in the distributed repository network. As soon as data relevant to him has changed in the repository network or new data has been added to the repository network, the manufacturer of the cosmetic product can start the validation process for his products. The monitoring of the distributed repository network or the starting of the validation process is accomplished by a client system assigned to the manufacturer.

As soon as the client system assigned to the manufacturer detects a change in the data stored in the distributed repository network, the client system checks which components of the cosmetic product are provided by an external supplier. In the exemplary embodiment shown in FIG. 7, such a component is provided by the supplier 1.

The client system then generates a first validation request message M1 and transmits it to the server system of the first supplier (supplier 1), the first validation request message M1 also being stored in the distributed repository network. The first validation request message M1 contains information about which data is to be validated by the first supplier's server system. In the present case, the first validation request message can include information about the fact that a specific component supplied by the first supplier must be validated, i.e. the ingredients regulated for this component should be communicated to the manufacturer or the client system.

The server of the first supplier receives the first validation request message M1 and transmits it to the trustlet, which is executed in the secure and trustworthy environment of the server.

The trustlet now analyzes the ingredients of the component to be validated, an ingredient in the present example being a component that is supplied by supplier 2 to supplier 1.

For all ingredients that are not supplied by another supplier, the trustlet of the first supplier's server checks whether they meet predetermined conditions. If an ingredient fulfills the predetermined condition, this ingredient must be communicated to the manufacturer of the cosmetic product or the client system. A predetermined condition can be, for example, that the ingredient is a regulated ingredient or ingredient that is subject to declaration. To check whether an ingredient satisfies such conditions, the trustlet carries out a comparison of the data of the ingredients with the data stored in the repository network, the trustlet determining whether data corresponding to the data of an ingredient are stored in the distributed repository network. In this case, the predetermined condition can be regarded as fulfilled if data records corresponding to the data of an ingredient are stored in the repository network.

The trustlet of the server of the first supplier now generates a first validation response message M2 and adds to this validation response message information about those ingredients that meet the predetermined conditions. Ingredients that do not meet the predetermined conditions are not added to the validation response message M2, so that they are not disclosed to the client or the manufacturer of the cosmetic product. The formulation of the component to be validated and requested with the first validation request message M1 therefore does not have to be disclosed and thus remains secret.

Before the validation response message M2 is transmitted to the client system, the server or the trustlet of the server of the first supplier generates a second validation request message M1′ in a corresponding manner for the components that are supplied by the second supplier and transfers this to the server of the second supplier.

The server of the second supplier accepts this second validation request message M1′ and transmits it to the server's trustlet, which is also executed in a secure and trustworthy area of the server. The trustlet of the second supplier's server also checks which ingredients of the requested component meet predetermined conditions, for example those that are subject to declaration or are regulated. The procedure here is analogous to the trustlet of the server of the first supplier. This means that the trustlet of the second supplier's server checks whether data corresponding to the ingredients of the component to be validated are stored in the distributed repository network.

The trustlet of the server of the second supplier now generates a second validation response message M2′ and adds to it information about those ingredients that meet the predetermined conditions. The ingredients that do not meet the predetermined conditions are not added to the validation response message M2′, i.e. they remain secret.

If the component to be validated by the trustlet of the second supplier does not have any ingredients that are supplied by another supplier, the trustlet transmits the validation response message M2′ to the server of the first supplier.

In the event that the component to be checked by the trustlet of the second supplier contains components that are supplied by another supplier, these components must be validated by a trustlet of the other suppliers, with the respective validation being analogous to that of the trustlets of the validations carried out by the first and the second supplier.

The first supplier's server receives the second validation response message M2′ from the second supplier's trustlet. The validation response message M2′ received from the server is transmitted to the server's trustlet. The trustlet of the first supplier's server can now insert the data received with the second validation response message M2′ into the first validation response message M2.

The trustlet then transmits the first validation response message M2 to the cosmetic product manufacturer's client system. The manufacturer of the cosmetic product now has all the information about the ingredients of the cosmetic product that are regulated or subject to declaration and that are contained in the components of the suppliers, so that the manufacturer can display information about this data on the packaging.

The manufacturer's client system now only has to validate the ingredients that are not contained in the components of the suppliers. For this purpose, a trustlet is also executed on the client system in a trustworthy and secure area of the client system. The validation of these ingredients is analogous to the validation that the trustlets carry out on the two server systems.

All validation request messages and validation response messages are stored in the distributed repository network and, if necessary, encrypted and signed.

Finally, the product data stored in the distributed repository network is marked as valid by the client system's trustlet.

With the help of the trustlets, which are executed according to embodiments of the invention, it can therefore be ensured that trustworthy data remain trustworthy even when a workflow is executed in a distributed system.

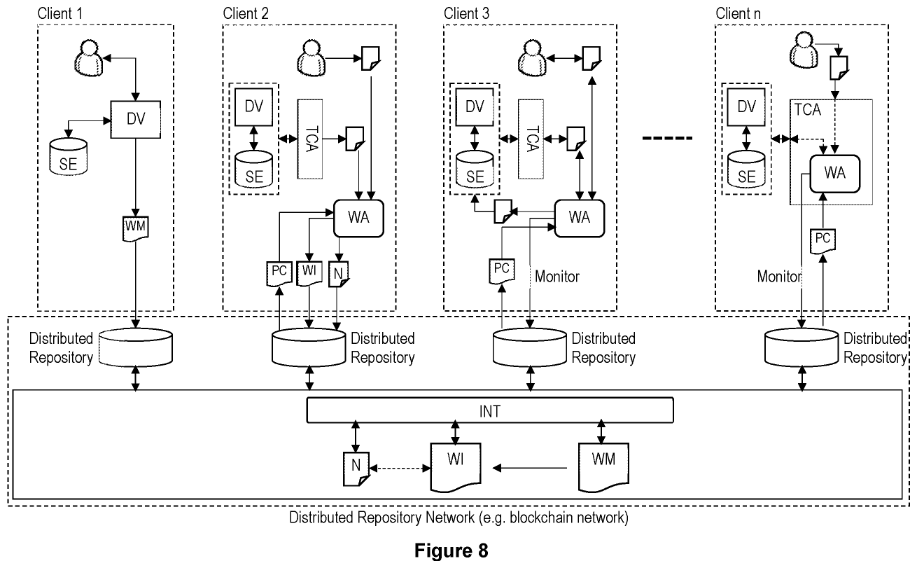

FIG. 8 shows a block diagram describing a further embodiment of the method for securely executing a workflow. The block diagram shown in FIG. 8 essentially corresponds to the block diagram shown FIG. 2. The description of FIG. 2 also applies to FIG. 8.

In contrast to FIG. 2, FIG. 8 shows an interpreter INT, which is stored as a smart contract in the distributed repository network and is executed there.

When executing a workflow instance, the interpreter on the one hand monitors the execution of the workflow instance, i.e. it monitors whether the workflow instance is being executed according to the workflow model.

On the other hand, the interpreter also takes over “control” of the workflow instance. This means that the interpreter generates a workflow instance from a workflow model, stores the generated workflow instance in the distributed repository network and generates events, tasks and notifications for each workflow step to be executed in a workflow instance and stores them in the repository network. FIG. 8 shows only one notification N generated by the interpreter INT as an example. Examples of events, tasks, and notifications are described with reference to FIGS. 3 and 4. In addition, the interpreter generates state data for each executed workflow step and stores this in the repository network.

The interpreter can also use the stored state data to check the current state of the workflow instance and the execution conditions of the workflow step for a workflow step before it generates the events, tasks and notifications for the workflow step.

What is claimed is:

1. A computer-implemented method for securely executing a workflow in a computer system comprising at least one distributed repository network and a number of client systems which are operatively coupled or operatively couplable to this repository network, wherein
a model of the workflow is stored in the repository network,
the workflow model comprises a number of workflow steps, a predetermined workflow step being defined as an initial step which is executed as the first step when executing the workflow in the computer system, and wherein stored in the workflow model for each workflow step are
execution conditions that must be met to execute the workflow step in the computer system,
events recorded in the computer system during the execution of the workflow step,
tasks assigned to a user, user role or client system when a workflow step is executed in the computer system, and
notifications made available to a user, user role or client system when a workflow step has been successfully executed in the computer system or when a workflow step has to be executed in the computer system,
to execute the workflow in the computer system a workflow instance is generated from the workflow model, the instance representing the workflow to be executed, the workflow instance being stored in the repository network,
when executing the workflow instance in the computer system,
first of all the initial step is executed, and then the further workflow steps are executed,
when a workflow step is started, the events, tasks and notifications are generated and stored in the repository network,
state data are stored in the repository network for each executed workflow step, and
a workflow application executed on at least one client system
receives notifications from the repository network that have been stored for a predetermined user in the repository network, and
executes a program code assigned to the workflow step for a workflow step to be executed on the at least one client system.

2. The method of claim 1, wherein information on a number of actions is stored in the workflow model for a workflow step, the actions being executed when the workflow step is executed, the actions being generated and stored in the repository network when the workflow step is started based on the information on the number of actions.

3. The method of claim 2, wherein
an interpreter is stored in the repository network,
the interpreter is executed in the repository network, and
when executing the workflow instance, the interpreter
monitors the execution of the workflow instance according to the associated workflow model,
when a workflow step is started, generates the events, tasks and notifications and stores them in the repository network according to the associated workflow model, and
stores the state data in the repository network for each executed workflow step.

4. The method of claim 3, wherein the interpreter is stored as a smart contract in the repository network.

5. The method of claim 1, wherein the program code assigned to a workflow step is stored in the repository network or on the client system as part of the workflow application.

6. The method of claim 1, wherein the status data includes a timestamp indicating the time the workflow step was executed, a user ID of the user who executed the workflow step, and a status of the workflow step.

7. The method of claim 1, wherein the distributed repository network is a blockchain network.

8. The method of claim 1, wherein when the program code assigned to the workflow step is executed by the workflow application, data are transmitted to the workflow application which are necessary for executing the program code.

9. The method of claim 8, wherein the transmitted data are stored in a storage device of the client system on which the program code is executed and/or in the distributed repository network.

10. The method of claim 1, wherein when the program code assigned to the workflow step is executed by the workflow application, data is generated, the data generated being stored in a storage device of the client system on which the program code is executed and/or in the distributed repository network.

11. The method of claim 10, wherein the data generated are stored in encrypted form in the repository network.

12. The method of claim 1, wherein the workflow model comprises a first and at least a second sub-workflow, each sub-workflow comprising a number of workflow steps, and wherein a model of the first sub-workflow is generated in a first client system of the number of client systems and a model of the at least a second sub-workflow is generated in a second client system of the number of client systems, the models of the sub-workflows being combined and stored in the distributed repository network as a workflow model.

13. The method of claim 12, wherein each model of a sub-workflow is assigned a user and/or a user role, the user and/or user role being granted management rights as a result of this assignment, in particular rights to change the respective model of the sub-workflow.

14. The method of claim 1, wherein before a workflow step is executed, the workflow application checks whether the execution conditions for executing the workflow step are met.

15. The method of claim 14, wherein the execution conditions include at least one from the group consisting of:
the workflow step to be executed must be a valid workflow step, wherein a workflow step to be executed is valid if preceding workflow steps on which the workflow step to be executed depends have been executed successfully,
the user who starts the workflow step to be executed must have permission to execute the workflow step, the permissions being stored in the distributed repository network, and
combinations thereof.
